(19) United States
(12) Patent Application Publication
(10) Pub. No.: US 2017/0143231 A1
(43) Pub. Date: May 25, 2017

(54) MONITORING THE BODY USING MICROWAVES

(71) Applicant: Safeetechnologies As, Tromsø (NO)

(72) Inventors: Bjørn Christian Østberg, Oslo (NO); Stephen Williams, London (GB); Svein Kjetil Jacobsen, Kvaløysletta (NO)

(21) Appl. No.: 15/320,577

(22) PCT Filed: Jun. 22, 2015
(86) PCT No.: PCT/EP2015/063988

§ 371 Date: Dec. 20, 2016

(30) Foreign Application Priority Data
Jun. 20, 2014 (GB) 1411063.9

(51) Int. Cl. A61B 5/05; A61B 5/00; A61B 5/03

(52) U.S. Cl. CPC A61B 5/0507; A61B 5/031; A61B 5/6803; A61B 5/0022; A61B 5/7278; A61B 5/7282; A61B 5/746; A61B 2505/01; A61B 2560/0431; A61B 2562/0228; A61B 2562/143

(57) ABSTRACT

An apparatus for determining characteristics of pulsatility of the brain (5) comprises: an ultra-wideband microwave transceiver (1) arranged to generate ultra-wideband microwave pulses (6); a transmitting means (3) arranged to transmit the ultra-wideband microwave pulses; and a receiving means (3) arranged to receive a signal corresponding to detection of the pulses. A method of determining characteristics of pulsatility of a first part of the brain comprises: transmitting ultra-wideband microwave pulses into the first part of the brain; receiving a signal corresponding to detection of the pulses; and processing the signal.

The present invention relates to a method and apparatus for non-invasive determination of physiological properties and conditions of tissue and organs of the body, for example the brain, in particular by measuring characteristics of the pulsatility of the brain.

Conventionally, damage to or pathologies of parts of the body, for example cerebral pathologies such as subarachnoid haemorrhage or stroke, may be identified by imaging the relevant part of the body (i.e. building up a visual map of the different tissues) using a magnetic resonance imaging (MRI) scan or using computerized tomography (CT).

MRI makes use of the property of nuclear magnetic resonance in the nuclei of atoms of the body in order to provide images, which can be generated in three dimensions. The apparatus is extremely bulky and heavy, and also requires very large, high-powered magnets. Such scanners are not portable. Thus, MRI scanners are limited in their use, in that they can only be used in dedicated units in medical facilities.

Furthermore, MRI scans require the patient to be placed in a confined space and to remain still for an extended length of time, of the order of between 15 and 90 minutes. In many cases, minimizing the time-to-treatment is essential for increasing the probability of a favourable outcome of treatment; the length of time taken for an MRI scan is not compatible with this aim.

CT scans make use of a source of ionizing radiation (for example, x-rays, gamma-rays or positrons). The ionizing radiation is detected after having passed through the body, to build up tomographic images of the area being scanned.

There are a number of disadvantages associated with CT scans. In particular, the use of ionizing radiation brings with it an increased risk of cancer. Additionally, CT scanners are heavy (even portable scanners are bulky and cumbersome to transport) and expensive.

There is therefore a need for an inexpensive and light-weight (highly portable) apparatus, and corresponding method, which can quickly detect whether there is a problem with the body, particularly the brain, and which does not expose the patient to ionizing radiation.

Recent advances in microwave imaging technology have shown potential for its use in non-destructive testing, including the field of medicine.

In such technologies, a microwave apparatus applies microwave radiation to image objects. The object to be imaged creates an effect on the microwave electromagnetic field when the radiation is transmitted through the object. Such changes of the electromagnetic field relate to attenuation, reflection and diffraction. These processes all depend on spatial variations of dielectric permittivity and conductivity of the object under investigation. Dielectric permittivity measures how much resistance is encountered when forming an electric field in a dielectric medium (an electrical insulator that can be polarized by an applied electric field). Conductivity on the other hand measures the material's ability to conduct such an electric current.

Attenuation is the gradual loss of intensity in various types of tissue due to dielectric propagation (the way microwaves travel in the electrical field). The induced changes in the complex permittivity of such types of tissue in turn create a reflection at the interfaces between different tissues. This reflection results in a combined back-propagating and partly transmitted wave at the specific site, i.e. diffraction.

Blood flow to the brain is not steady; it varies in step with the heartbeat. Since the brain is enclosed within a confined volume (the skull), the changing blood pressure causes the brain to pulsate. This characteristic of the brain is known as pulsatility, or intracranial pulsation. The normal resting adult human heart rate ranges from 60 to 80 beats per minute. Following exercise or stress, the heart may beat much faster. Therefore, the frequency of brain pulsatility is of the order of 1 Hz or below.

The pulsatility of the brain can be affected by various medical conditions, especially cerebral pathologies such as subarachnoid haemorrhage or stroke, for example. Therefore, measurement of the characteristics of the pulsatility of the brain may be used an in indicator of these (and other) cerebral pathologies.

According to a first aspect of the present invention, there is provided a method of determining characteristics of pulsatility of a first part of the brain comprising: transmitting microwave pulses into the first part of the brain; receiving a signal corresponding to detection of the pulses; and processing the signal.

The detected pulses may be reflected pulses which have been reflected from the first part of the brain, or may be transmitted pulses which have been transmitted through the first part of the brain.

Preferably, the microwave pulses are ultra-wideband microwave pulses. Here, “ultra-wideband” has the standard meaning known in the art, i.e. a pulse for which the emitted signal bandwidth exceeds the lesser of 500 MHz or 20% of the centre frequency. Ultra-wideband pulses are particularly suitable as they may be short range (for example, propagating up to about 10 cm in the body) and may have low power (for example, −10 to −30 dBm).

Preferably, the characteristic that is determined is the frequency of pulsatility (pulsation) of the first part of the brain. Alternatively or additionally, the characteristic that is determined is the amplitude of pulsatility (pulsation) of the first part of the brain. Alternatively or additionally, the characteristic may be the pattern or the characteristics of motion of the brain tissue.

Preferably, the method further comprises determining the characteristics of the pulsatility of a second part of the brain and comparing it to the characteristics of the pulsatility of the first part of the brain. Here, the first part and second part of the brain may be any two spatially separated regions in the brain. For example, the first part of the brain may be a portion of one of the hemispheres of the brain, and the second part of the brain may be a portion of the opposed hemisphere of the brain. Alternatively, the first part of the brain may be a portion of the frontal (anterior) area of the brain, and the second part of the brain may be a portion of the rear (posterior) area of the brain.

The comparison between the characteristics of the pulsatility of a second part of the brain may be used to indicate the presence or absence of a particular brain pathology. For example, following a traumatic head injury to the one part of the brain, bleeding into the skull may affect the pulsatility of the injured part. A comparison between the pulsatility of the injured part and a non-injured part may allow the presence or absence of a particular brain pathology to be determined.

Alternatively, the characteristics of the pulsatility of the first part of the brain may be compared against an expected value of the characteristic, which is, for example, derived from a medical study, for example, an analysis of a sample of healthy patients, a mathematical model, or an earlier measurement of the characteristics of the pulsatility of the first part of the brain taken for the same patient.

Thus, these embodiments are based on the recognition that pulsatility in the underlying cerebral tissue reflects the well-being of the brain, whereas perturbations in the detected characteristics (for example amplitude and frequency) may represent an abnormal pathological state, for example, haemorrhage, stroke or a mass lesion. Here “perturbations” refers either to the difference between the expected value of the characteristic (known from previous medical studies, for example) and what is actually measured, or to the difference between the measured characteristic in different parts of the brain.

Preferably, the ultra-wideband microwave pulses have a broadband frequency of between 0.5 GHz and 10 GHz. Each pulse may have a duration of the order of 1 nanosecond. The pulse may be generated by an impulse radar transceiver. The impulse radar transceiver may be provided as a single-chip integrated circuit.

A reduced bandwidth will degrade the longitudinal (along beam) resolution, and so it is preferable to maximize the bandwidth to maximize the information content of the pulses. The lower bound of the frequency may be limited by antenna size and diffraction effects, and the upper bound of the frequency may be dictated by losses in the propagation medium.

Antenna dimension and transmitted wavelength are highly coupled. At below 0.5 GHz, known antennae become omnidirectional (i.e. it becomes a point source and is no longer a proper antenna). In order to obtain a well-focused beam, either a large antenna aperture or a small wavelength (high frequency) must be used. Such a small wavelength to antenna dimension ratio is represented by a laterally narrow antenna beam. For example, for a given practical antenna size of 30-40 mm, the lowest applicable wavelength would be 0.5 GHz.

Additionally, the efficiency of the antennae known for similar applications degrades below 0.5 GHz, such that the antenna mainly produces heat or reflects the incoming signal back to the transmitter.

In applications where a larger antenna would be acceptable, use of frequencies lower than 0.5 GHz is possible.

The upper limit of the broadband frequency is governed by the degree of attenuation of high frequencies of microwave radiation in body tissue. For example, muscle, which has a high water content, is prone to a high loss of intensity, with losses increasing above 3-4 GHz at a fast rate. Thus, depending on the observation depth of the body tissue, wave information content in pulses above 10 GHz is lost even for small propagation distances (on the order of centimetres).

In embodiments where it is desirable to scan deeply into the brain, (for example, to a depth of 50 to 60 mm), the upper part of the spectrum (from approximately 7 to 10 GHz) may be omitted, as the power of these frequencies will be absorbed by the tissue.

Preferably, the signal corresponding to detection of the pulses includes a plurality of samples for each pulse. That is, the reflected or transmitted signal may be sampled a plurality of times. This is referred to herein as “fast-time” sampling. Fast-time sampling may be on the time scale of nanoseconds or below, for example from 1 picosecond to 1 nanosecond.

The fast-time samples may be taken sequentially in time. Each may comprise a measured amplitude for the reflected or transmitted signal, which has been detected at the time at which the sample has been taken. The time taken for a signal to be detected will depend on the distance travelled by the signal. Thus, each sample may comprise an amplitude measurement from a different depth along the signal axis. That is, fast-time signals correspond to distance (depth, range) into the brain, along the signal axis.

The largest amplitudes may be measured at depths corresponding to depths within the skull at which there is an interface between different tissues, causing a relatively strong reflection from that point. Thus, fast-time signals may be used to locate the depth of the outer part of the brain for each pulse.

In order to measure the characteristics of pulsatility of the brain, pulse-to-pulse variations may be considered. This is referred to herein as “slow-time” sampling. Slow-time sampling may be on the time scale of milliseconds, for example from 1 millisecond to 500 milliseconds.

The time resolution for slow-time sampling is dependent on the pulse repetition rate (the slow-time sampling rate).

Preferably, the ultra-wideband microwave pulses have a pulse repetition rate of greater than 5 Hz, more preferably greater than 10 Hz, and most preferably 20 Hz or greater. Collecting data with a higher pulse repetition rate may provide data with higher sensitivity and may allow for the signal-to-noise ratio to be improved for the same scanning time. However, this must be balanced with the consideration that additional processing power is necessary to handle the increased pulse repetition rate.

Preferably, the ultra-wideband microwave pulses have a pulse repetition rate of less than 150 Hz, more preferably less than 100 Hz, and most preferably 50 Hz or less.

Preferably, at least one pulsatility cycle should be measured. So to get a measurement of a 1 Hz pulsatile signal, (60 bpm), at least one second of scanning is required. Scanning for longer may provide data with higher sensitivity. Thus, receiving a signal corresponding to detection of the pulses may be carried out for 5 seconds or more, more preferably 10 seconds or more, and most preferably 20 seconds or more. Preferably, receiving a signal corresponding to detection of the pulses may need to be carried out for 1 minute or less.

Preferably, the method comprises processing the signal with respect to fast-time (for each pulse) and pulse-to-pulse variations (slow-time). Fast-time sampling may enable the location of a given interface (for example, the outer layer of the brain) to be determined for each pulse, and the slow-time sampling may enable the change in location (for example, movement due to pulsatility) of the given interface to be measured as a function of time. That is, slow-time sampling may be used to measure the dynamics of the brain. The frequency of pulsatility may therefore be determined.

The spatial amplitude of pulsatility (that is, how much the brain moves) may be measured by finding the sample number which corresponds to the peak of the pulsatile signal, and the sample number which corresponds to the trough of the pulsatile signal. The difference between these corresponds to a distance that can be calculated, if the time between each sample is known, and the speed of microwaves in tissue is also known.

Brain dynamics may be derived from the data set by an adequate method capable of extracting periodic signal components in the range from 1 to 3 seconds. The method is preferably robust against interfering internal and external noise and may be able to discriminate between data containing medical information and signal variations stemming from inherent drift in the transceiver electronics.

Thus, the decoupling of low frequency pulsatile medical information from the slow drift in the transceiver electronics may be obtained by advanced algorithms.

Preferably, the signal is processed using Principal Component Analysis (PCA). However, algorithms other than PCA may be used instead. For example, one or more of the following processes or algorithms may be used in addition to or instead of PCA: filtering (band-pass/Butterworth, etc.), FFT (fast Fourier transform), ICA (independent component analysis), non-linear regression, power spectral density algorithms.

PCA attempts to decompose a multivariate signal into independent non-Gaussian signals. In cases where that the statistical independence assumption of PCA is correct (as is assumed here), blind PCA separation of a mixed signal into its components is a relatively robust method for moderate signal-to-noise ratios.

In such an analysis, the measurement for one transceiving antenna configuration may be represented as a 2-D matrix X(i,j) with dimensions M×N. The index T denotes fast-time (or distance into the brain), which may be on the nanosecond scale, whereas T denotes the pulse-to-pulse slow-time index. Using one antenna, only a measurement in the depth dimension can be made. To build up a measurement in three dimensions, additional information is necessary. This may be provided by providing an array of spatially separated antennae, and/or by mechanical movement of an antenna or array or antennae, to provide measurements in two additional dimensions. Thus, information related to the lateral dimensions of the head may be added to the data matrix giving the generalized 4-D representation X(i,j,k,l) where the indices ‘k’ and ‘l’ account for cross range coordinates. Using some kind of localization algorithm, e.g. delay-and-sum, the brain dynamics may be pin-pointed to a 3D spatial position within the head (not only to a certain depth, as is possible with a single stationary antenna).

The PCA analysis may find a set of variables that explain as much as possible of the variance in all samples while being uncorrelated with each other. The first six components together might explain something like 99% of all the data. Two of these (at least) may reflect the pulsatile signal: one representing the frequency at peak and one at the valley. The frequency will be the same, but data will be different. Each component may be checked for a pulsatility signal in the expected range of 0.5-2 Hz. The pulsatility signal may then be analysed to determine characteristics of pulsatility (such as amplitude and frequency).

The present invention makes use of the properties of diffraction and reflection of microwave radiation to study characteristics of the brain, particularly the pulsatility of the brain. The present invention does not rely on an image of the brain being generated. Rather, the present invention may be thought of as providing a simple detection system.

Advantageously, the invention may be used to complement more established technologies (such as CT and MRI scans) by allowing the rapid assessment of brain health and/or functioning in the field (for example, at the site of an emergency). The potential to monitor healthy brain function and physiology as well as changes in brain pulsation following trauma such as a subarachnoid haemorrhage or stroke are envisaged.

Though the method of the first aspect relates to a method of determining characteristics of the pulsatility of the brain, the invention is seen as being more generally applicable. Thus, according to a second aspect of the present invention, there is provided a method of determining characteristics of a first part of the brain comprising: transmitting microwave pulses into the first part of the brain; receiving a signal corresponding to detection of the pulses; and processing the signal.

Preferably, the microwave pulses are ultra-wideband microwave pulses.

Disorders where organic pathology leads to changes in the properties of the reflected signal may lead to a novel fingerprint for specific cerebral pathologies. Integration of these data with other measures may lead to greater specificity and sensitivity in clinical assessment. The invention may therefore also find clinical utility in, for example, paediatric and dementia assessment.

The large bandwidth and high sensitivity of the ultra-wideband microwave sensors to ultra-low power signals make the ultra-wideband microwave radar principle suitable for more general medical applications, including mobile and continuous monitoring of vital functions in the human body (for example, lung function/movement, or arterial function). The speed, resolution and potential sensitivity may provide an alternative or complementary modality to existing technologies.

Thus, even more generally, according to a third aspect of the present invention, there is provided a method of determining characteristics of tissue or organs of the body comprising: transmitting microwave pulses into the body; receiving a signal corresponding to detection of the pulses; and processing the signal.

Preferably, the microwave pulses are ultra-wideband microwave pulses.

The preferred features of the first aspect as set out above relate equally to the second and third aspects, where applicable. In particular, the analysis comprising slow-time and fast-time sampling (as set out above) in combination with the second and third aspects is particularly preferable.

Thus, preferably, the method of the second and third aspects comprises processing the signal with respect to fast-time (for each pulse) and pulse-to-pulse variations (slow-time). Fast-time sampling may enable the location of a given interface to be determined for each pulse, and the slow-time sampling may enable the change in location of the given interface to be measured as a function of time.

The invention also extends to an apparatus configured to perform the methods discussed above. Thus, according to a fourth aspect of the present invention, there is provided an apparatus comprising: a microwave transceiver arranged to generate microwave pulses; a transmitting means arranged to transmit the microwave pulses; and a receiving means arranged to receive a signal corresponding to detection of the pulses.

Preferably, the microwave pulses are ultra-wideband microwave pulses. The apparatus may be suitable for, or may be operable to, determine characteristics of the pulsatility of the brain. More generally, the apparatus may be for, or may be operable to, determine characteristics of the brain. Even more generally, the apparatus may be for, or may be operable to, determine characteristics of tissue or organs of the body.

The apparatus may comprise a processing unit operable to process the signal to determine characteristics of pulsatility of the brain. Alternatively, the apparatus may comprise a communications unit for communication with a remote processing unit via a communications network. In this embodiment, a remote server may comprise a processing unit which is operable to process the signal to determine characteristics of pulsatility of the brain.

Preferably, the apparatus comprises a plurality of ultra-wideband microwave units, each comprising an ultra-wideband microwave transceiver, an ultra-wideband microwave transmitting antenna and an ultra-wideband microwave receiving antenna. That is, the transmitting antenna and receiving antenna are different antennae which are separately provided.

Alternatively, the apparatus comprises a plurality of ultra-wideband microwave units, each comprising an ultra-wideband microwave transceiver, and an ultra-wideband microwave transmitting/receiving antenna. In this case a coupler may also be provided.

The apparatus may comprise a support structure to which the plurality of ultra-wideband microwave units are attached. Preferably, the plurality of units are arranged so as to direct microwave radiation to spatially separated (and preferably opposed) regions of the brain. The regions may for example be the front and the back of the brain, or opposite hemispheres of the brain.

The support structure may include a coupling medium for coupling to the part of the body to be examined, for example, a coupling medium for coupling to the skull. This may be integrated into a suitable housing. It will be appreciated that such a unit may be easily portable.

Thus, in a preferred embodiment, the support structure is arranged to conform to the relevant part of the body. Thus, in the case of an apparatus to investigate the brain, it may be in the form of a helmet. This may be designed as a medical apparatus which is placed on the patent's head in the event of trauma or illness, or it may be a helmet that would be worn for other reasons. Such a helmet may be a helmet to be worn during leisure pursuits, sport or travel. Some non-limiting examples include motorcycle helmets, ski helmets, boxing helmets, helmets for American football, etc. The apparatus may be retrofitted to an existing helmet, or the helmet may be purpose-built to include the apparatus.

Alternatively, the support structure may be a hand-held device. Such a device may not be capable of directing ultra-wideband microwave radiation to spatially separate parts of the body (for example the brain). In that case, in use, the hand-held device may be placed in a first position relative to the body part to be examined, and a first measurement may be made, and then the hand-held device may be moved and placed in a second, different position to make a second measurement of the body part to be examined at the second position.

The apparatus may comprise a warning indicator. Preferably, the processing unit is operable to control the warning indicator to output a warning if the characteristics of pulsatility of the brain are outside of a predetermined range. The warning indicator may output light and/or sound. For example, the warning indicator may be a speaker. The warning indicator may be an LED which may flash.

Preferably the antenna(e) is/are a micro strip patch antenna(e). The thickness of the antennae may be less than 5 mm, preferably less than 2 mm, and most preferably 1 mm or less. Here, the thickness of the antenna refers to the distance between the ground plane and upper surface plane of the micro strip patch antenna.

The invention also extends to an apparatus of the fourth aspect (which also may incorporate any of the preferred features thereof described above) for carrying out the method of the first, second or third aspects (which may incorporate any of the preferred features thereof described above).

The method or the apparatus of any of the above described aspects of the invention (or the preferred features thereof) may be combined with existing techniques (such as ultrasound, near Infrared spectroscopy, EEG, CT and/or MR) and the radar data may be processed in combination with one or more of the mentioned techniques to enhance sensitivity and specificity of the whole (combined) system.

Certain preferred embodiments will now be described by way of example only and with reference to the accompanying drawings, in which:

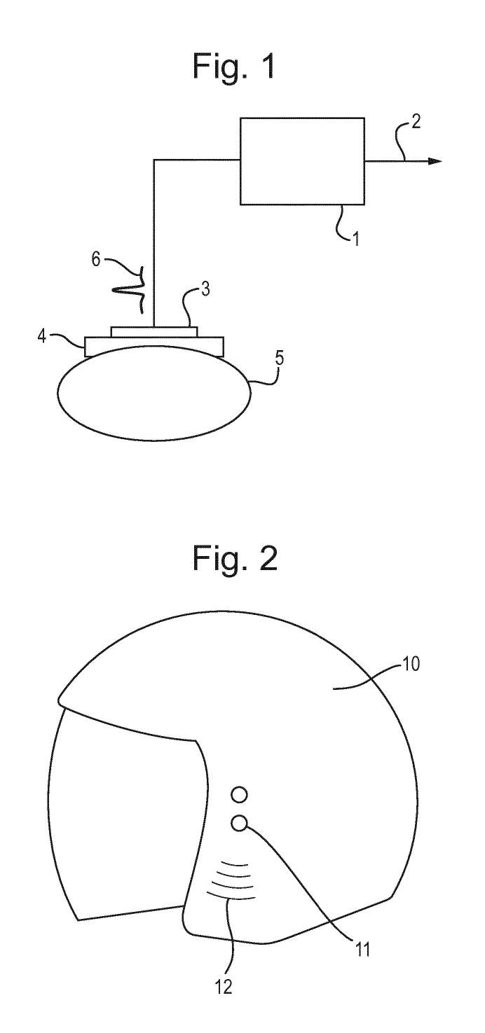

FIG. 1 shows a schematic view of an ultra-wideband microwave unit for use in an apparatus in accordance with an embodiment of the present invention;

FIG. 2 shows an apparatus in accordance with an embodiment of the present invention;

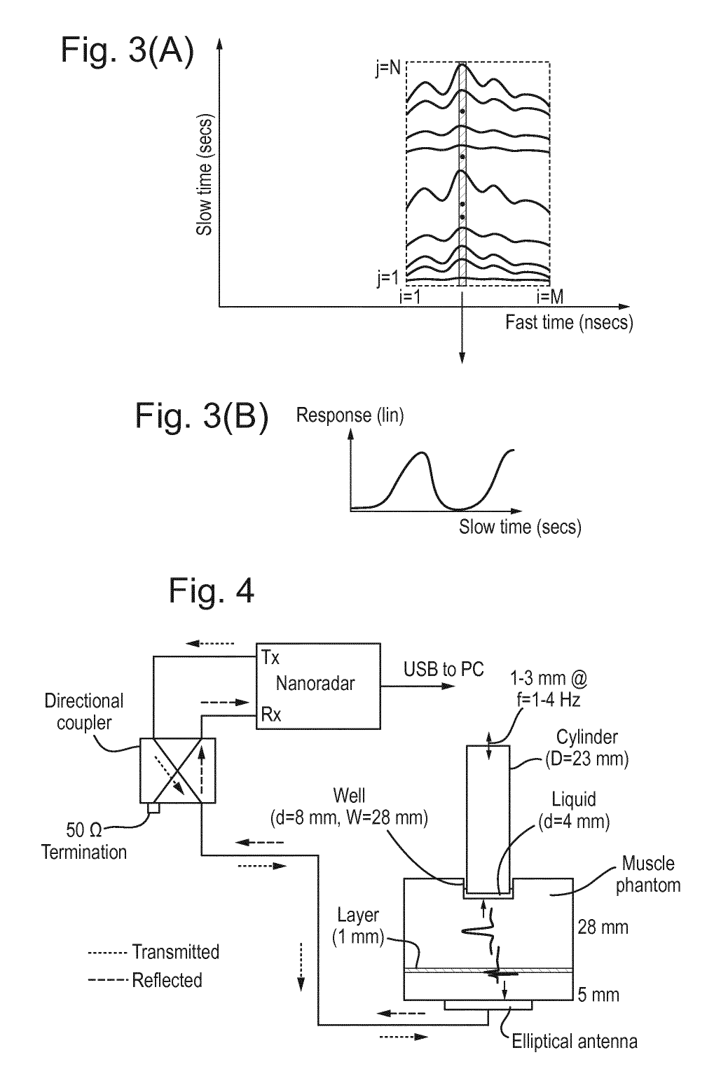

FIG. 3 shows an exemplary 2-D data matrix for one transceiving antenna of the apparatus of FIG. 1;

FIG. 4 shows an experimental test set-up; and

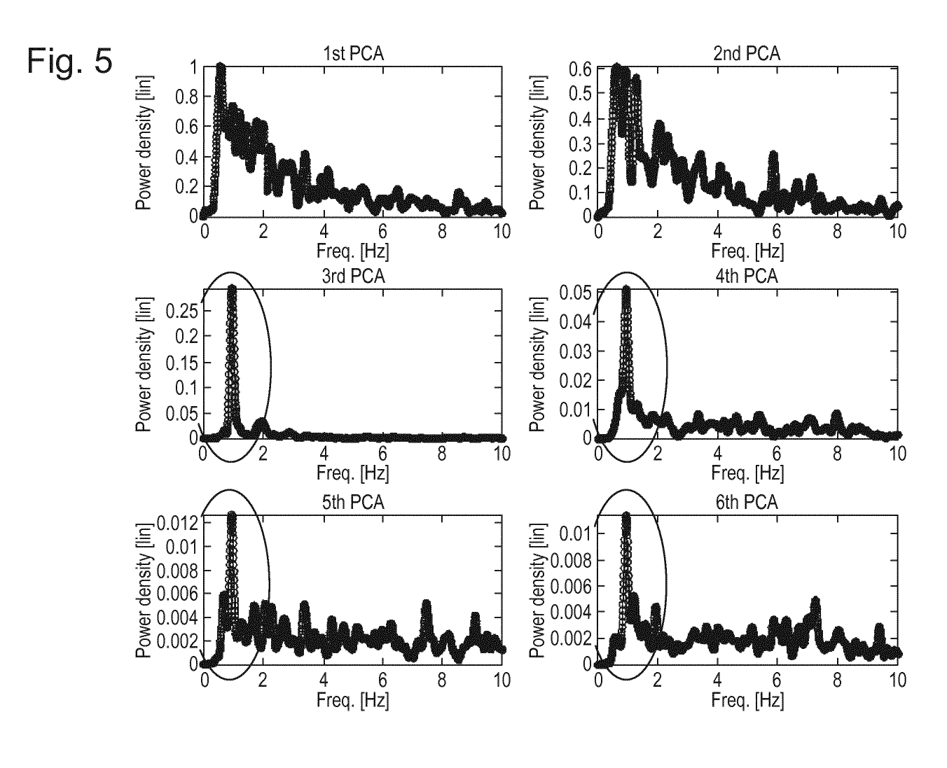

FIG. 5 shows power density as a function of frequency for the first to sixth components following an independent component analysis in the test set-up of FIG. 4.

FIG. 1 shows: an ultra-wideband transceiving microwave and data processor unit 1; a connection means 2 for connection to a post-processing unit (not shown); a ultra-wideband antenna 3; and a coupling medium 4. An ultra-wideband pulse 6 is transmitted into the brain 5.

The ultra-wideband transceiving microwave radar 1 is a fully integrated nano-scale impulse radar transceiver with a single-chip impulse-based radar designed for low-power (−20 dBm) high-performance applications. Such a radar provides a low-cost, highly integrated and highly robust solution for a wide range of remote sensing applications and could employ 32-bit digital integration and 512 parallel samplers for maximum frame depth and sensitivity, as well as a fully programmable frame offset for an extensive detection range. Non-limiting examples of such a units are the XeThru X1 (previously NVA6100) and X2 (previously NVA6201) single-chip impulse radar transceiver integrated circuits (CMOS chips) provided by Novelda AS. The ultra-wideband microwave radiation pulse 6 is emitted with a frequency using sinusoid antennae from 3 to 6 GHz, with a pulse repetition rate of 20 Hz.

The ultra-wideband antenna 3 is a transmitting/receiving micro patch antenna.

The coupling medium 4 ensures coupling (minimal wave reflection) and prevents the beam from diverging (as it will in air) for a given aperture dimension.

FIG. 2 shows an apparatus including the unit of FIG. 1, integrated into a motorcycle helmet 10. The helmet comprises a plurality of units supported by and spaced around the helmet, so that microwave radiation can be directed from separate units to each of the two hemispheres of the brain, and to the front and back of the brain. This allows comparison of the pulsatility of the two hemispheres, and independently, comparison of the pulsatility of the front and back of the brain.

The helmet 10 includes warning indicators 11 and 12. The first indicator 11 comprises LEDs, which emit light (for example, a red flashing light) if it is determined that the brain has been damaged. The second indicator 12 comprises a speaker which can emit sound if it is determined that the brain has been damaged.

The helmet 10 also comprises a communications system (not shown) for communication via a telecommunications network to a server (not shown), which analyses data from the microwave unit to determine whether the brain is damaged. If such a determination is made, a signal is sent from the server to the helmet to activate the warning indicators 11, 12.

Post-processing of the data obtained by the microwave unit is carried out by the remote server. The signal is processed with respect to fast-time (on a time scale of the order of nanoseconds) and pulse-to-pulse variations (on a timescale of the order of milliseconds).

The experimental setup shown in FIG. 4 comprises a transceiving radar system with separate Tx and Rx inputs/outputs and a directional coupler included for monostatic antenna operation. The antenna is coupled to a layered lossy load consisting of a coupling medium (5 mm thick), a skull medium (1 mm layer) and a 28 mm muscle phantom mimicking brain tissue. Pulsatile variations are simulated by moving a target cylinder (23 mm diameter) within a square liquid-filled well of 28 mm at 26 mm depth in the phantom.

The liquid and the cylinder material were varied to obtain combinations with more or less reflection contrast for the incoming electromagnetic pulse. Longitudinal movement of the cylinder was in the range of 1-2 mm, with a periodicity of 1 second to 0.25 seconds.

The measurement for the transceiving antenna configuration is represented as a 2-D matrix X(i,j) with dimensions M×N. The index T denotes fast-time (or distance into the target) on the nanosecond scale, whereas T denotes the pulse-to-pulse slow-time index (on the scale of seconds).

Such a matrix is shown in FIG. 3A. Fast-time corresponds to range along the radar beam and slow-time samples the pulse-to-pulse variations due to dynamics of the target. The shaded band corresponds to a given depth and the black dots illustrate the pulsatile variations sampled on a slow-time scale.

FIG. 3B shows a schematic of the pulsatile signal (although this is highly simplified as in the real case it will be buried in noise or other more dominant signals e.g. receiver system gain variations). The pulsatile signal is expected to be similar to a pulse train giving a spectral response with a dominant frequency and less marked, but detectable, higher harmonics. The pulsatile signal shown in FIG. 3B is extracted from the matrix using Principal Component Analysis. The M principal components of the data matrix X are given by:

Y=A^{T}X
Here, X=[x̅₁̅,x̅₂̅ . . . x̅_̅{̅M̅}̅]^{T}, and is the zero-mean input data (M×N)
and Y=[y̅₁̅.y̅₂̅ . . . y̅_̅{̅M̅}̅]^{T}, and is the output matrix of principal components.
   A can be computed using the covariance matrix,

$${C}_{X} = \frac{1}{N} {XX}^{T}$$

The next step is to find the eigenvalue and eigenvector matrices of Cₓ, [symbol] and Φ.

Δ=diag(λ₁, λ₂ . . . λ_{M}) where λ₁, λ₂ . . . λ_{M} are the eigenvalues.

After arranging the eigenvalues in decreasing order, A is given by:

A=[Φ̅₁̅, Φ̅₂̅ . . . Φ̅_̅{̅M̅}̅

The principal component matrix S is given by:

S=A^{T}X

S=[s̅₁̅, s̅₂̅ . . . s̅_̅{̅M̅}̅]^{T}

The s̅ vectors are principal components arranged in strength of variance.

The result of such an independent component analysis, carried out using the test-set up shown in FIG. 4, is shown in FIG. 5. Here, a signal at 1 Hz is clearly visible in the 3^{rd} to 6^{th} components.

1. A method of determining characteristics of pulsatility of a first part of the brain comprising:
transmitting ultra-wideband microwave pulses into the first part of the brain;
receiving a signal corresponding to detection of the pulses; and
processing the signal.

2. A method according to claim 1, wherein the characteristic that is determined is the frequency and/or amplitude of pulsation of the first part of the brain.

3. A method according to claim 1 or 2, comprising comparing the measured characteristics of the pulsatility of the first part of the brain to the expected characteristics of the pulsatility of the first part of the brain.

4. A method according to claim 1 or 2, comprising determining the characteristics of the pulsatility of a second part of the brain and comparing it to the characteristics of the pulsatility of the first part of the brain.

5. A method according to claim 4, wherein the first part of the brain is a portion of one of the hemispheres of the brain, and the second part of the brain is a portion in the opposed hemisphere of the brain.

6. A method according to claim 4, wherein the first part of the brain is the frontal (anterior) portion of the brain, and the second part is the posterior portion of the brain.

7. A method according to any preceding claim wherein the ultra-wideband microwave pulses have a broadband frequency of between 0.5 GHz and 10 GHz.

8. A method according any preceding claim wherein the ultra-wideband microwave pulses have a pulse repetition rate of greater than 5 Hz, more preferably greater than 10 Hz, and most preferably 20 Hz or greater, and/or wherein the ultra-wideband microwave pulses have a pulse repetition rate of less than 150 Hz, more preferably less than 100 Hz, and most preferably 50 Hz or less.

9. A method according to any preceding claim comprising processing the signal with respect to fast-time sampling within a single pulse and slow-time pulse-to-pulse variations.

10. A method according to any preceding claim, wherein the signal is processed using Principal Component Analysis.

11. A method according to any preceding claim, wherein the ultra-wideband microwave pulses are transmitted using an impulse radar transceiver.

12. An apparatus for determining characteristics of pulsatility of the brain comprising:
an ultra-wideband microwave transceiver arranged to generate ultra-wideband microwave pulses;
a transmitting means arranged to transmit the ultra-wideband microwave pulses; and
a receiving means arranged to receive a signal corresponding to detection of the pulses.

13. An apparatus according to claim 12, comprising a processing unit operable to process the signal to determine characteristics of pulsatility of the brain.

14. An apparatus according to claim 12, comprising a communications unit for communication with a remote processing unit, preferably via a telecommunications network.

15. An apparatus according claim 13 or 14, wherein the apparatus comprises a warning indicator, and wherein the processing unit is operable to control the warning indicator to output a warning if the characteristics of pulsatility of the brain are outside of a predetermined range.

16. An apparatus according to any of claims 12 to 15, comprising a plurality of ultra-wideband microwave units, each comprising an ultra-wideband microwave transceiver, an ultra-wideband microwave transmitting antenna and an ultra-wideband microwave receiving antenna.

17. An apparatus according to any of claims 12 to 15, comprising a plurality of ultra-wideband microwave units, each comprising an ultra-wideband microwave transceiver, and an ultra-wideband microwave transmitting/receiving antenna.

18. An apparatus according to claim 16 or 17, wherein the antenna(e) is/are micro strip patch antenna(e).

19. An apparatus according to claim 16, 17, or 18 comprising a support structure to which the plurality of ultra-wideband microwave units are attached.

20. An apparatus according to claim 19 wherein the support structure includes a coupling medium to the skull.

21. An apparatus according to claim 19 or 20, wherein the support structure is a helmet, and preferably the plurality of units are arranged so as to direct microwave radiation to opposed regions of the brain.

22. An apparatus according to claim 18, 19 or 20, wherein the support structure is a hand-held device.

23. An apparatus according to any of claims 12 to 22, wherein the ultra-wideband microwave transceiver is an impulse radar transceiver.

24. An apparatus according to any of claims 12 to 23, for carrying out the method of any of claims 1 to 11.

25. An apparatus substantially as described herein, with reference to the accompanying drawings.

26. A method substantially as described herein, with reference to the accompanying drawings.
